(19) United States
(12) Patent Application Publication
(10) Pub. No.: US 2005/0175686 A1
(43) Pub. Date: Aug. 11, 2005

(54) Drug delivery system using a solubilized gelatin shell composition and unit dose drug delivery using a special shape soft gelatin capsule

(76) Inventors: Ramachandran Radhakrishnan, Bangalore (IN); Nehru Babu Gaddipati, Somerset, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/776,418

(22) Filed: Feb. 11, 2004

(51) Int. Cl. A61K 9/48; A61K 9/64

(52) U.S. Cl. 424/456

(57) ABSTRACT

Disclosed is an improved drug delivery device for delivering a fill pharmaceutical composition. The drug delivery device comprises a soft gelatin capsule having a shell comprising gelatin and plasticizer wherein the shell is dissoluble upon dispersion into warm water. The invention also discloses snip-off or twist-off soft gelatin capsules offering unit dose convenience. The composition of the shell used in constructing the soft gelatin capsules comprises gelatin in the range of approximately 40% to 48% and a plasticizer ranging in amount from approximately 14% to 25%.

### BACKGROUND OF THE INVENTION

1. Field of the Invention

This invention in general relates to Improved soft gelatin device containing a therapeutically effective amount of pharmaceutical fill composition wherein the device comprises a twist-off or snip-off end on one side. More particularly, the present invention relates to a soft gelatin capsule having an improved shell composition that enables the capsule shell to quickly dissolve in warm water, thus releasing the medication to provide a solution or suspension.

2. Description of the Related Art

Oral drug administration necessitates devising drug administration devices that helps achieving various therapeutic objectives. These objectives include bioavailability, patient compliance, shelf life and so on. Of the above, patient compliance is an important issue that has prompted devising drug delivery devices that helps patients to easily consume the drug. Currently available solid dosage forms utilized to administer medication as a solution or suspension include: i) soluble tablets, ii) quick dissolve tablets, iii) effervescent tablets, iv) effervescent powders, v) soluble powders and dry powder suspensions. However, there are no softgel capsules designed to readily dissolve in water to form a solution or suspension. The gelatin shell from the conventional soft gelatin capsules swells and do not readily dissolve in warm water. Soft gelatin capsules belonging to this category of drug administration systems comprise of water-soluble gelatin shell containing a liquid or semi solid inner fill. An active ingredient can be incorporated into the outer shell, the inner fill or both.

Patient compliance with softgel formulations is improved because soft gelatin capsules' soft, elastic character makes them easier to swallow than conventional tablets or hard gelatin capsules. Furthermore, since the dosage form is generally swallowed, it is unnecessary to flavor or otherwise mask any unpleasant taste of the active pharmaceutical ingredients. Finally, unlike tablets, soft gelatin capsules do not chip or powder. In view thereof, soft gelatin drug delivery device are a preferred mode of drug administration.

In general, gelatin capsules shell formulations of soft gelatin capsules consist of gelatin and one or more ingredients, which are added as plasticizers to produce a capsule to the desired hardness. Typical plasticizers include Glycerin, Sorbitol, and Anidrisorb 85/70 (Anidrisorb 85/70 is an aqueous solution of D-Sorbitol and Sorbitans). In certain formulations drug-gelatin; drug-glycerin interactions result in case hardening, poor disintegration/dissolution of the drug molecule.

U.S. Pat. No. 5,985,321 to Brox, et al. describes a soft gelatin capsule, which comprises a shell, and a liquid filling, wherein said shell comprises gelatin, plasticizers and, if required, further auxiliary agents. In the process for making the soft gelatin capsule, the gelatin bands are cooled with a liquid, and preferably with water.

U.S. Pat. No. 5,614,217 to Chiprich, et al. describes a gelatin shell encapsulating the fill material, the gelatin capsule comprising about 30% to about 50% gelatin, about 5% to about 15% by weight water, about 15% to 35% by weight nialtitol syrup, and about 8% to about 30% by weight elasticity reducing gelatin extender, like starch, amylose starch, esterified starch.

U.S. Pat. No. 5,641,512 to Cimiluca, et al. describes a soft gelatin shell composition that contains a xanthine derivative, such as caffeine and also comprises from about 20% to about 60% gelatin, more preferably from about 25% to about 50% gelatin, and most preferably from about 40% to about 50% gelatin. The gelatin can be of Type A & Type B, or a mixture thereof with bloom numbers ranging from about 60 to about 300, about 10% to about 35% plasticizer, preferably from about 10% to about 25% plasticizer, and most preferably from about 10% to about 20% plasticizer, preferably glycerin, from about 15% to about 50% water, more preferably from about 25% to about 40% water, and most preferably from about 30% to about 40% water.

U.S. Pat. No. 4,820,524 to Berta, et al. describes a novel capsule-like medicament, method for producing such medicaments and apparatus thereof. The method provides a procedure for coating solid cores, such as caplets, with gelatinous coatings to produce a shiny, capsule-like medicament. Such medicaments are achieved by individually dipping and drying first one end, and then the other end of each caplet to provide a coating, which is smoother and easier to swallow than an uncoated caplet. The production of these capsule-like medicaments is readily facilitated by simple and inexpensive modifications, which can be made to existing empty gelatin capsule making equipment.

U.S. Pat. No. 5,681,606 and U.S. Pat. No. 5,871,798 to Hutchison, et al. describes about method of preparing a water-based beverage, wherein the capsular shell composition, comprises 38% to 58% of glycerol as a plasticizer and other additive 15% of unbleached starch acetate. Invention also describes a shell enclosing an additive added to a potable aqueous liquid comprising water heated to 70° C.

Patient compliance is an important aspect of drug delivery. To a very great extent, soft gelatin capsules support patient compliance. However, some patients do not prefer to swallow gelatin capsules, and some patients, as pediatric and geriatric populations, have difficulty in swallowing. Sometimes an immediate action of the medication is required.

In this invention an improved soft gelatin capsule having a shell composition, which enables the capsule to dissolve in warm water in less than two minutes, has been disclosed. This in turn helps patients to easily dissolve the capsule in a glass of water and consume the same, or the softgel capsule contents can be squeezed out to dissolve in a glass of water or fruit juice and a palatable formulation can be administered by emptying the capsule contents directly into the patient's mouth.

### SUMMARY OF THE INVENTION

In accordance with one embodiment, there is provided an improved drug delivery device comprising a soft gelatin capsule and methods for producing the same. A soft gelatin drug delivery device for administering pharmaceutical formulations comprising a soft gelatin capsule having a shell that enables quick dissolution of the capsule in warm water has been described. The quick dissolution property of soft gelatin device is because of its shell composition. The shell composition enables dissolution relatively quickly in water or physiological fluids (gastric and intestinal fluid).

In accordance with another embodiment, the drug delivery system described is a soft gelatin capsule with a special gelatin shell composition that dissolves in warm water completely, thereby releasing the capsule contents to form a solution or suspension in water, which can be easily swallowed. This drug delivery device system can be utilized to administer medication for geriatric and pediatric population that cannot swallow medication in capsule or tablet.

In accordance with another embodiment, the drug delivery system described is snip-off or twist-off soft gelatin capsule where the capsule contents can be squeezed out to dissolve or disperse the drug in water or fruit juice.

In accordance with another embodiment, the drug delivery system described is snip-off or twist-off softgels offering unit dose convenience where the capsule containing liquid or semisolid fills formulated with taste/odor masking formulas, the contents are swallowed by directly squeezing of the capsule contents into the patient's mouth and this type of drug delivery system doesn't require water for swallowing and is quite useful while traveling.

In accordance with yet another embodiment, the drug delivery system can be swallowed similar to the conventional soft gelatin capsule and includes excipients as sweeteners, and flavoring agents in the gelatin shell, fill or both.

In accordance with yet another embodiment, there is the development of a gelatin shell that readily breaks open at body temperature 37° C. which is also the temperature used for in vitro testing media such as disintegration and dissolution medium.

In accordance with one preferred embodiment there are provided shell composition for a soft gelatin device, said composition comprising 38.0-46.0% by weight of Gelatin, 14-25% by weight of Sorbitol Solution 70% (non crystallizable), 0.2-0.6% by weight of Glycine, 0.02-0.03% by weight of Butylated Hydroxy Anisole and 40.5-45.5% by weight of purified water.

In accordance with another preferred embodiment there are provided shell composition for a soft gelatin device, said composition comprising 38.0-46.0% by weight of Gelatin, 14-25% by weight of Sorbitol Solution, 70% (non crystallizable), 0.2-0.6% by weight of Glycine, 0.02-0.03% by weight of Butylated Hydroxy Anisole, 0.02-0.03% by weight of Butylated Hydroxy Toluene, 40.5-45.5% by weight of Purified water.

In accordance with another preferred embodiment there are provided shell composition for a soft gelatin device, said composition comprising 38.0-46.0% by weight of Gelatin, 14-25% by weight of Sorbitol Solution, 70%, 0.2-0.6% by weight of Glycine, 0.02-0.03% by weight of Butylated Hydroxy Anisole, 0.42-0.46% by weight of Citric acid, 40.5-45.5% by weight of Purified water.

In accordance with another preferred embodiment there are provided shell composition for a soft gelatin device, said composition comprising 38.0-46.0% by weight of Gelatin, 14-25% by weight of Sorbitol Solution, 70%, 0.2-0.6% by weight of Glycine, 0.02-0.03% by weight of Butylated Hydroxy Anisole, 0.02-0.03% by weight of Butylated Hydroxy Toluene, 0.42-0.46% by weight of Citric acid, 40.5-45.5% by weight of Purified water.

In accordance with another preferred embodiment there are provided soft gelatin capsule filled with fill composition containing a therapeutically effective amount of pharmaceutical actives selected from a group consisting of Ibuprofen, Pseudoephedrine HCl, Naproxen Sodium, Acetaminophen and mixtures thereof.

### DETAILED DESCRIPTION OF THE PREFERRED EMBODIMENT

Currently soft gelatin capsules are used for swallowing internally and no unit dosage softgels are available for pharmaceutical use. Use of a snip off or twist-off softgels offers the advantage of either dissolving or dispersing the contents in water or fruit juice. It will also offer the advantage of delivering the formulations directly into the patient's mouth and does not require the aid of water for swallowing. This type of drug delivery system is particularly useful when the patients are traveling, and thus also helps patient compliance.

Alternatively, the gelatin capsules are manufactured with a twist-off or snip-off end on one side. The gelatin capsule contents can be emptied into a container and dissolved or dispersed in water. The gelatin capsules are formulated with taste masking and flavoring agents in the fill preparation, and the capsule contents can be emptied directly into patient's mouth and the empty gelatin capsule shell is disposed off.

The present invention relates to an improved soft gelatin drug device. The invention also provides a shell composition for use in constructing soft gelatin capsules includes gelatin in the range of approximately 40% to 48% by weight and a plasticizer ranging in amount from approximately 16% to 35% by weight. A preferred Plasticizer for use with the preferred Capsule or shell formulation includes a non-crystallizing Sorbitol solution. When Sorbitol Solution 70% (non crystallizing) and Anidrisorb 85/70 are used alone as the plasticizers, the amount preferably ranges from approximately 16% to 35% by weight. Capsule formulations can also include other suitable additives such as anti-oxidants, amino acids, sweeteners, flavoring agents and coloring agents, which are utilized to impart specific characteristics including capsule aesthetics.

Below are examples illustrating several soft gelatin shell compositions made in accordance with the present invention. The examples presented below illustrate particular embodiment of the invention and is not intended to limit the scope of the specification.

### EXAMPLE 1

| | Percentage |
|---|---|
| Ingredient | by weight |
| Gelatin | 38.0-46.0 |
| Sorbitol Solution, 70% (non crystallizable) | 14-25 |
| Glycine | 0.2-0.6 |
| Butylated Hydroxy Anisole | 0.02-0.03 |
| Purified water | 40.5-45.5 |

### EXAMPLE 2

| | Percentage |
|---|---|
| Ingredient | by weight |
| Gelatin | 38.0-46.0 |
| Sorbitol Solution, 70% (non crystallizable) | 14-25 |
| Glycine | 0.2-0.6 |
| Butylated Hydroxy Anisole | 0.02-0.03 |
| Butylated Hydroxy Toluene | 0.02-0.03 |
| Purified water | 40.5-45.5 |

### EXAMPLE 3

| Ingredient | Percentage by weight |
|---|---|
| Gelatin | 38.0-46.0 |
| Sorbitol Solution, 70% | 14-25 |
| Glycine | 0.2-0.6 |
| Butylated Hydroxy Anisole | 0.02-0.03 |
| Citric Acid | 0.42-0.46 |
| Purified water | 40.5-45.5 |

### EXAMPLE 4

| Ingredient | Percentage by weight |
|---|---|
| Gelatin | 38.0-46.0 |
| Sorbitol Solution, 70% | 14-25 |
| Glycine | 0.2-0.6 |
| Butylated Hydroxy Anisole | 0.02-0.03 |
| Butylated Hydroxy Toluene | 0.02-0.03 |
| Citric Acid | 0.42-0.46 |
| Purified water | 40.5-45.5 |

Below are examples illustrating several soft gelatin Fill compositions made in accordance with the present invention. The examples presented below illustrate particular embodiment of the invention and is not intended to limit the scope of the invention.

### EXAMPLE 1

| Ingredient | Milligrams per capsule |
|---|---|
| Ibuprofen | 200 to 400 |
| Potassium Carbonate | 4 to 10 |
| Yellow Bees Wax | 4 to 10 |
| Soybean Oil | 12 to 200 |
| Purified Water | 5 to 15 |
| Sucaralose | 10 to 30 |
| Natural Lemon Oil | 1 to 10 |
| Polysorbate 80 | 15 to 40 |
| Citric Acid | 5 to 15 |

### EXAMPLE 2

| Ingredient | Milligrams per capsule |
|---|---|
| Pseudoephedrine HCl | 30 to 60 |
| Yellow Bees Wax | 5 to 20 |
| Lecithin | 3 to 10 |
| Silicon Dioxide Colloidal | 3 to 10 |
| Soybean Oil | 100 to 250 |
| Partially Hydrogenated Vegetable Oil | 10 to 25 |
| Sucaralose | 5 to 30 |
| Natural Lemon Oil | 1 to 10 |

### EXAMPLE 3

| Ingredient | Milligrams per capsule |
|---|---|
| Naproxen Sodium | 200 to 550 |
| Yellow Bees Wax | 5 to 20 |
| Lecithin | 5 to 20 |
| Soybean Oil | 200 to 800 |
| Partially Hydrogenated Vegetable Oil | 15 to 40 |
| Sucaralose | 20 to 60 |
| Natural Lemon Oil | 1 to 10 |

### EXAMPLE 4

| Ingredient | Milligrams per capsule |
|---|---|
| Acetaminophen | 200 to 325 |
| Pseudoephedrine HCl | 30 |
| Dextromethorphan HBr | 10 |
| Polyethyleneglycol 400 | 500 to 700 |
| Propyleneglycol | 30 to 50 |
| Povidone K-30 | 60 to 75 |
| Sucaralose | 30 to 60 |
| Natural Lemon Oil | 1 to 10 |

### EXAMPLE 5

| Ingredient | Milligrams per capsule |
|---|---|
| Acetaminophen | 200 to 325 |
| Pseudoephedrine HCl | 30 |
| Dextromethorphan HBr | 10 to 15 |
| Doxylamine Succinate | 6.25 |
| Polyethyleneglycol 400 | 500 to 700 |
| Propyleneglycol | 30 to 50 |
| Povidone K-30 | 60 to 75 |
| Sucaralose | 30 to 60 |
| Natural Lemon Oil | 1 to 10 |

Certain modifications and improvements of the disclosed invention will skilled in the art without departing from the scope of invention, which is the appended claims.

1. An improved drug delivery device, comprising:
a soft gelatin capsule having a shell comprising gelatin and plasticizer wherein the shell readily breaks open upon dispersion into a medium.

2. The drug delivery device according to claim 1, wherein the medium in which the shell readily breaks open upon dispersion is warm water or hot water.

3. The drug delivery device according to claim 1, wherein the shell readily breaks open when exposed to 37° C. temperature in liquid medium.

4. The drug delivery device according to claim 1, wherein the drug delivery device is snip-off or twist-off soft gelatin capsule.

5. The drug delivery device according to claim 1, wherein the shell is made from a shell composition comprising about 38% to 48% gelatin by weight and about 16% to 35% plasticizer by weight.

6. The drug delivery device according to claim 5, wherein the plasticizer comprises sorbitol solution 70% (non crystallizable) and is present at about 14 to 25% by weight.

7. The drug delivery device according to claim 6, wherein the shell composition further comprises:
0.2-0.6% by weight of Glycine;
0.02-0.03% by weight of Butylated Hydroxy Anisole; and
40.5-45.5% by weight of purified water.

8. The shell composition according to claim 7, wherein the shell composition further comprises 0.02-0.03% by weight of Butylated Hydroxy Toluene.

9. The shell composition according to claim 8, wherein the shell composition further comprises 0.42-0.46% by weight of Citric acid.

10. An improved gelatin shell composition in a soft gelatin drug delivery device wherein the shell readily breaks open upon dispersion into a medium.

11. The drug delivery device according to claim 10, wherein the medium in which the shell readily breaks open upon dispersion is warm water.

12. The drug delivery device according to claim 11, wherein the shell readily breaks open when exposed to 37° C. temperature in liquid medium.

13. A shell composition for use in a soft gelatin capsule, the shell composition comprising gelatin and plasticizer wherein the soft gelatin capsule readily breaks open upon dispersion into a medium.

14. The drug delivery device according to claim 13, wherein the medium in which the shell readily breaks open upon dispersion is warm water.

15. The drug delivery device according to claim 13, wherein the shell readily breaks open when exposed to 37° C. temperature in liquid medium.

16. The shell composition according to claim 13, wherein the shell comprises gelatin in the range of approximately 40% to 48% by weight.

17. The shell composition according to claim 13, wherein the shell comprises plasticizer in the range of approximately 14% to 25% by weight.

18. The shell composition according to claim 16, wherein the shell comprises approximately 38% to 46% by weight of gelatin.

19. The shell composition according to claim 18, wherein the shell composition further comprises:
14-25% by weight of Sorbitol Solution 70% (non crystallizable);
0.2-0.6% by weight of Glycine;
0.02-0.03% by weight of Butylated Hydroxy Anisole; and
40.5-45.5% by weight of purified water.

20. The shell composition according to claim 18, wherein the shell composition further comprises:
14-25% by weight of Sorbitol Solution, 70% (non crystallizable),
0.2-0.6% by weight of Glycine,
0.02-0.03% by weight of Butylated Hydroxy Anisole,
0.02-0.03% by weight of Butylated Hydroxy Toluene, and
40.5-45.5% by weight of Purified water.

21. The shell composition according to claim 18, wherein the shell composition further comprises:
14-25% by weight of Sorbitol Solution, 70%,
0.2-0.6% by weight of Glycine,
0.02-0.03% by weight of Butylated Hydroxy Anisole,
0.42-0.46% by weight of Citric acid, and
40.5-45.5% by weight of Purified water.

22. The shell composition according to claim 18, wherein the shell composition further comprises:
14-25% by weight of Sorbitol Solution, 70%,
0.2-0.6% by weight of Glycine,
0.02-0.03% by weight of Butylated Hydroxy Anisole,
0.02-0.03% by weight of Butylated Hydroxy Toluene,
0.42-0.46% by weight of Citric acid, and
40.5-45.5% by weight of Purified water.

23. An improved drug delivery device, comprising a soft gelatin capsule having a shell, which readily breaks open upon dispersion into a medium wherein, the capsule comprises a therapeutically effective amount of pharmaceutical actives.

24. The drug delivery device according to claim 23, wherein the pharmaceutical actives are selected from a group consisting of Ibuprofen, Pseudoephedrine HCl, Naproxen Sodium, Acetaminophen and mixtures thereof.

25. The drug delivery device according to claim 23, wherein the medium in which the shell readily breaks open upon dispersion is lukewarm water.

26. The drug delivery device according to claim 23, wherein the shell readily breaks open when exposed to 37° C. temperature in liquid medium.
